(19) United States
(12) Patent Application Publication
(10) Pub. No.: US 2025/0281665 A1
(43) Pub. Date: Sep. 11, 2025

(54) BIONIC DRESSING-FORMING COMPOSITION, BIONIC DRESSING, AND METHOD OF FORMING THE BIONIC DRESSING

(71) Applicant: THE HONG KONG POLYTECHNIC UNIVERSITY, Hong Kong (CN)

(72) Inventors: Xin ZHAO, Hong Kong (CN); Yuhe YANG, Hong Kong (CN); Di SUO, Hong Kong (CN); Tianpeng XU, Hong Kong (CN)

(21) Appl. No.: 18/964,799

(22) Filed: Dec. 2, 2024

(30) Foreign Application Priority Data
Mar. 6, 2024 (CN) 202410252800.6

(51) Int. Cl. A61L 26/00

(52) U.S. Cl. CPC A61L 26/0052; A61L 26/0066; A61L 26/0076; A61L 26/008; A61L 2300/404; A61L 2300/418; A61L 2300/608; A61L 2400/04

(57) ABSTRACT

A bionic dressing-forming composition, a bionic dressing, and a method of forming the bionic dressing. The bionic dressing-forming composition herein includes an aqueous phase and an oil phase, wherein the oil phase includes a hydrophobic prepolymer or a hydrophobic monomer, the hydrophobic prepolymer or the hydrophobic monomer has a first photopolymerizable functional group, and the oil phase has an intrinsic viscosity lower than 10⁶ mPa·s at ambient temperature; and the aqueous phase includes a hydrophilic hydrogel prepolymer or a hydrophilic hydrogel monomer and an active ingredient, and the hydrophilic hydrogel prepolymer or the hydrophilic hydrogel monomer has a second photopolymerizable functional group, the aqueous phase has an intrinsic viscosity lower than 10⁶ mPa·s at ambient temperature. The first photopolymerizable functional group and the second photopolymerizable functional group includes a —C≡C— bond and are capable of polymerization and/or crosslinking initiated by light. Upon application of the composition, the oil phase and the aqueous phase are capable of forming a bilayer and forming a bionic dressing with a double-layer crosslinked structure of hydrophobic polymer and hydrophilic hydrogel.

### CROSS-REFERENCE TO RELATED APPLICATIONS

The present application claims priority from China Patent Application No. 202410252800.6, filed on Mar. 6, 2024, which is hereby incorporated by reference in its entirety.

### TECHNICAL FIELD

The present disclosure generally relates to a bionic dressing-forming composition, a bionic dressing, and a method of forming a bionic dressing.

### BACKGROUND

Skin is an elastic, multi-layered tissue and the largest organ in the human body. Skin has the function of sensing and protecting the body from physical damage and pathogens. Extensive trauma with acute and uncontrolled bleeding can pose a real threat to survival. Although wound dressings have made great progress, inappropriate dressing selection and wound management can still easily lead to infection, difficulty in healing, and even scar formation, which places a huge burden on both patients and the health system.

Clinically, various dressings have been widely used in wound management. Among all dressings, hydrogel-based products, such as PIVOT (alginate) and Simpurity™ (polyvinyl alcohol), have outstanding advantages of maintaining a moist repair microenvironment, absorbing tissue exudate, and stimulating the extracellular matrix (ECM). Thus, they are considered to be superior to other materials (such as cotton sponge, rubber and foam)

However, currently available commercial hydrogel dressings usually lack rapid hemostatic properties and cannot be applied to extensive emergency wounds with uncontrollable bleeding. Furthermore, current hydrogel dressings also exhibit limited wound adhesion and mechanical properties against external interference. They also suffer from problems, such as excessive water evaporation and inevitable microbial infections, which can lead to delayed wound healing. More importantly, the severe vascular damage in extensive burns can substantially hinder the blood supply, causing depletion of nutrients and delay in regenerative cell (e.g., fibroblasts) rescue, leading to deferred tissue regeneration. In addition, scar-free repair has always been one of the goals pursued by the medical community, but current hydrogel dressings may cause obvious scar formation, affecting the patient's appearance and quality of life. In summary, current hydrogel dressings lack adequate protection, cleaning, and nourishment of the wound site. Therefore, improved dressings that can accelerate wound hemostasis and healing while inhibiting scar hyperplasia is needed.

### SUMMARY

One of the objectives of the present disclosure is to provide a bionic dressing-forming composition that can conveniently form a bionic dressing which can achieve rapid hemostasis of wounds, and at the same time provide a moist and sterile environment, which is conducive to achieving scarless healing of wounds.

In a first aspect, the present disclosure provides a bionic dressing-forming composition comprising an aqueous phase and an oil phase, wherein
the oil phase comprises a hydrophobic prepolymer or a hydrophobic monomer, the hydrophobic prepolymer or the hydrophobic monomer has a first photopolymerizable functional group, and the oil phase has an intrinsic viscosity lower than 106 mPa·s at ambient temperature, such as 1-1000 mPa·s, 10-200 mPa·s, 5-100 mPa s or 1-80 mPa s;
the aqueous phase comprises a hydrophilic hydrogel prepolymer or a hydrophilic hydrogel monomer and an active ingredient, and the hydrophilic hydrogel prepolymer or the hydrophilic hydrogel monomer has a second photopolymerizable functional group, the aqueous phase has an intrinsic viscosity lower than 106 mPa·s at ambient temperature, such as 1-1000 mPa·s, 20-200 mPa·s, 10-100 mPa·s, 5-50 mPa·s or 1-30 mPa·s;
the first photopolymerizable functional group and the second photopolymerizable functional group comprises a —C═C— bond and are capable of polymerization and/or crosslinking initiated by light, and
upon application of the composition, the oil phase and the aqueous phase spontaneously form a bilayer thereby forming a bionic dressing with a double-layer cross-linked structure comprising a hydrophobic polymer and a hydrophilic hydrogel.

In certain embodiments, the first photopolymerizable functional group and the second photopolymerizable functional group in the bionic dressing-forming composition of the present disclosure comprise one or more of acrylate, methacrylate, acrylamide, methacrylamide, styrene, N-vinylpyrrolidone, hydroxybutyl vinyl ether, diethylene glycol divinyl ether or phenyl glycidyl ether.

In certain embodiments, the hydrophobic prepolymer or the hydrophobic monomer comprises one or more of poly (lactic acid-propylene glycol-lactic acid) dimethyl acrylate, polypropylene glycol diacrylate, polypropylene glycol dimethacrylate, polypropylene acrylate and polymethyl methacrylate.

In certain embodiments, the composition further comprises a first photoinitiator for initiating polymerization and/or crosslinking reaction of the hydrophobic prepolymer or the hydrophobic monomer, wherein the first photoinitiator can comprise bis(2,4,6-trimethylbenzoyl) phenyl phosphine oxide or lithium phenyl-2,4,6-trimethylbenzoyl phosphinate.

In certain embodiments, the hydrophilic hydrogel prepolymer or the hydrophilic hydrogel monomer comprises one or more of methacrylated gelatin, acrylated gelatin, methacrylated hyaluronic acid, acrylated hyaluronic acid, methacrylated chitosan, acrylated chitosan and hydrophilic polyethylene glycol diacrylate.

In certain embodiments, the composition further comprises a second photoinitiator for initiating polymerization and/or crosslinking reaction of the hydrophilic hydrogel prepolymer or the hydrophilic hydrogel monomer, wherein the second photoinitiator can comprise 2-hydroxy-1-[4-(2-hydroxyethoxy)phenyl]-2-methyl-1-acetone, or camphorquinone.

In certain embodiments, the active ingredient in the aqueous phase is selected from a hemostatic active ingredient, such as a water-soluble calcium salt (such as CaCl₂) or thrombin; a wound healing active ingredient, such as a growth factor; and combinations thereof.

In certain embodiments, the concentration of the active ingredient in the aqueous phase is 0.1-70 wt/wt %, 5-30 wt/wt %, or 5-10 wt/wt %.

In certain embodiments, the oil phase and/or the aqueous phase further comprises an antimicrobial active ingredient.

In certain embodiments, the antimicrobial active ingredient is selected from triclosan and curcumin.

In certain embodiments, the concentration of the antimicrobial active ingredient in the oil phase is 0.1-20 wt/wt %, 1-10 wt/wt %, or 2-7 wt/wt %.

In certain embodiments, the composition does not comprise an emulsifier or a surfactant.

In certain embodiments, the composition forms a bionic dressing with a double-layer cross-linked structure within 100 seconds, 80 seconds, 60 seconds, 50 seconds, 40 seconds, 30 seconds, 20 seconds, 10 seconds, 5 seconds or 1 second after photoinitiation.

In certain embodiments, the composition is in the form of a liquid formulation. In certain embodiments, the liquid formulation is in the form of a spray.

In certain embodiments, the composition comprises: an oil phase comprising poly (lactic acid-propylene glycol-lactic acid) dimethacrylate and triclosan; an aqueous phase comprising gelatin methacryloyl and Ca²⁺.

In a second aspect of the present disclosure, provided is a bionic dressing formed from the bionic dressing-forming composition as described above, the bionic dressing comprises at least one double-layer structure having a skin conforming layer and an outer layer, the outer layer is adjacent the skin conforming layer, wherein the skin conforming layer is in contact with the skin and comprises a hydrophilic hydrogel, and the outer layer comprises a hydrophobic polymer. In the above bionic dressing, the hydrophilic hydrogel is formed from an aqueous phase comprising a hydrophilic hydrogel prepolymer or a hydrophilic hydrogel monomer and an active ingredient, and the hydrophobic polymer is formed from an oil phase comprising a hydrophobic prepolymer or a hydrophobic monomer.

In certain embodiments, the bionic dressing of the present disclosure is formed by the following process: mixing the oil phase and the aqueous phase to form a mixture including the oil phase and the aqueous phase, applying the mixture to a surface to form an uncured bionic dressing and irradiating the uncured bionic dressing with light to form the bionic dressing.

In a third aspect of the present disclosure, provided is the use of the bionic dressing-forming composition as described above in preparing a bionic dressing.

In a fourth aspect of the present disclosure, provided is a method of forming the above-mentioned bionic dressing, the method comprises: applying a mixture including the aqueous phase and the oil phase of the bionic dressing-forming composition as described above; and curing the mixture via polymerization and/or crosslinking initiated by light, thereby obtaining the bionic dressing.

The above technical solution of the present disclosure has at least the following advantages.
1. The bionic dressing of the present disclosure has a skin conforming layer that can conform to skin wounds, and can achieve rapid hemostasis of wounds and firmly adhere to the wound site and adapt to joint movements. The outer layer of the bionic dressing can provide the strength required for the dressing and isolate the wound site from the external environment, thereby maintaining a moist and sterile environment at the wound site, which is conducive to achieving scarless healing of wounds.
2. The bionic dressing of the present disclosure allows multiple material components to be loaded in both the skin conforming layer and the outer layer to match the cascade process of wound healing and meet the precise medical needs of different wound conditions. For example, Ca²⁺ can be included in the skin conforming layer, allowing the layer to activate the coagulation cascade, further facilitating rapid hemostasis; the outer layer can be loaded with triclosan (TCS) to enhance the antimicrobial effect.
3. Both the oil phase and the aqueous phase of the bionic dressing-forming composition provided by the present disclosure comprise photo-crosslinking materials, so they can be rapidly polymerized and/or crosslinked in situ during the application process, and the interface between the two layers can have strong interfacial force to prevent interlayer peeling.
4. The bionic dressing-forming composition of the present disclosure can be applied by a spray method, which is convenient for application on irregular or large-area wounds. This feature can quickly reduce pain and the risk of infection, and is easy and fast to use. In particular, the hydrogel-containing skin conforming layer that can be formed in situ has more outstanding advantages than existing preformulated hydrogels, including its excellent portability and flexibility, rapid application and high conformability to irregular wounds.

### BRIEF DESCRIPTION OF THE DRAWINGS

The above and other objects and features of the present disclosure will become apparent from the following description of the present disclosure when taken in conjunction with the accompanying drawings.

FIG. 1 is a schematic diagram (A-B) of the material composition and application process of a spray-type bionic dressing-forming composition provided by an example of the present disclosure;

FIG. 2 shows the test result of (A) Fourier transform infrared spectrum and (B) ¹H nuclear magnetic resonance spectrum to verify the chemical structures of gelatin methacryloyl (GelMA) and poly (lactic acid-propylene glycol-lactic acid) dimethacrylate (PGLADMA).

FIG. 3 shows the spontaneous water/oil separation in the presence of different mass fractions of calcium chloride (CaCl₂) after vigorous mixing of GelMA (in the aqueous phase) and PGLADMA (in the oil phase);

FIG. 4 is a macroscopic view of a double-layer anti-scar dressing formed by photo-crosslinking in the presence of calcium chloride (CaCl₂) with different mass fractions, and a scanning electron microscope view of the material and the interface between the two layers;

FIG. 5 is a diagram showing the strong tissue adhesion and joint movement adaptability of the bionic dressing of the present disclosure;

FIG. 6 is a (A-B) schematic diagram of the mechanical property test results of the bionic dressing of the present disclosure and (C) the single layer formed by the oil phase or the aqueous phase respectively;

FIG. 7 shows the homeostatic performance test results of the bionic dressing of the present invention using the rat tail truncated model as an example, where (A) photos of the hemostatic effect of the bionic dressing of the present disclosure; (B) quantification of clotting time; (C) the relative blood loss ratio calculated by weighing the blood-stained filter paper; (D) the amount of bleeding observed by photos of filter paper absorbing effusion of blood.

FIG. 8 shows an (A-B) evaluation result of the microbicidal ability of the oil phase (photo-crosslinkable PGLADMA+ triclosan) of the bionic dressing of the present invention, taking E. coli and S. aureus as examples;

FIG. 9 shows the (A-F) biocompatibility evaluation result of the oil phase (photo-crosslinkable PGLADMA+ triclosan) and the aqueous phase (GelMA+ calcium chloride solution) in the bionic dressing-forming composition of the present disclosure, wherein CL+ means the present of collagenase, and CL-means the absent of collagenase;

FIG. 10 shows the (A) evaluation results of in vitro angiogenesis ability of the aqueous phase (GelMA+ calcium chloride solution), the microscopic fluorescence diagram and (B) the quantitative evaluation of newly formed vascular branches, taking human umbilical vein endothelial cells (HUVEC) as an example;

FIG. 11 shows an (A-E) evaluation of the healing effect of the bionic dressing of the present disclosure based on the area change and healing ratio of the wound, taking the full-thickness skin wound model of rats infected by Staphylococcus aureus as an example;

FIG. 12 shows an (A-F) evaluation of the therapeutic effect of the double-layer bionic dressing on scar formation by Masson staining and immunofluorescence staining of collagen in rat skin tissue.

FIG. 13 shows the bionic dressings (BDMs) in accordance with certain embodiments described herein enhanced wound healing in full-thickness porcine skin wound model. (A) Schematic illustration showing the construction of S. aureus-infected full-thickness porcine skin wound model and the representative wound images treated with different wound masks. (B) H&E staining of wound sections after 28-day treatment. The black arrow lines indicated the width of granulation tissue. (C) Masson's trichrome staining of wound sections after 28-day treatment. The dash line indicated the wound areas. (D) Luminal structure formation and (E) laser Doppler perfusion images of the healing area. The black arrow indicated the vessels. Quantification of (F) wound area percentage, (G) granulation tissue width and (H) blood vessel density of the healing area. Sample size n=5 for all experiments by a one-way or two-way ANOVA with a Tukey's post hoc test for multiple comparisons. Data are presented as mean #SD. *p<0.05 and ***p<0.001 are considered statistically significant.

FIG. 14 shows quantification of (A) collagen occupied area in Masson's trichrome staining and (B) flux intensity in laser Doppler perfusion imaging. Sample size n=5 for all experiments by a one-way or two-way ANOVA with a Tukey's post hoc test for multiple comparisons. Data are presented as mean±SD. *p<0.05 and ***p<0.001 are considered statistically significant.

### DETAILED DESCRIPTION

### Definitions

Throughout the present application, where a composition is described as having, including, or comprising a particular component, or where a process is described as having, including, or comprising a particular process step, it is contemplated that the composition taught herein may also consists essentially of or consists of listed components, and the processes taught herein may also consists essentially of or consists of listed process steps.

In the present application, when an element or component is said to be included in and/or selected from a list of recited elements or components, it should be understood that the element or component can be any one of the recited elements or components, or the element or component can be selected from the group consisting of two or more recited elements or components. Furthermore, it should be understood that elements and/or features of the compositions or methods described herein may be combined in various ways, whether explicitly or implicitly expressed herein, without departing from the spirit and scope of the teachings of the present invention.

It should be understood that the order of steps or the order in which certain actions are performed is not critical so long as the present teachings remain operable. Additionally, two or more steps or actions can be performed simultaneously.

Unless expressly stated otherwise, use of the singular herein includes the plural (and vice versa). Furthermore, when the term “about” is used before a numerical value, the present teachings also include the specific numerical value itself, unless specifically stated otherwise. As used herein, the term “about” refers to a variation of ±10%, ±7%, ±5%, ±3%, ±1% or ±0% from the nominal value unless otherwise stated or inferred.

In the present disclosure, the terms “optional” or “optionally” refer to embodiments in which the features defined by the term (such as components, steps, etc.) may or may not be present.

The “ambient temperature” condition mentioned in the present invention refers to general temperature or room temperature, which is usually defined as a temperature condition of 20-25° C., for example, 25° C.

The advantages and features of the present disclosure will become more apparent from the following optimal embodiments and illustrative examples. The scope of the disclosure is not limited to any specific embodiments described herein.

The present disclosure provides a bionic dressing-forming composition comprising an aqueous phase and an oil phase, wherein

The disclosure provides a bionic comprises a hydrophobic prepolymer or a hydrophobic monomer, the hydrophobic prepolymer or the hydrophobic monomer has a first photopolymerizable functional group, and the oil phase has an intrinsic viscosity lower than 106 mPa·s at ambient temperature; and the aqueous phase comprises a hydrophilic hydrogel prepolymer or a hydrophilic hydrogel monomer and an active ingredient, and the hydrophilic hydrogel prepolymer or the hydrophilic hydrogel monomer has a second photopolymerizable functional group, the aqueous phase has an intrinsic viscosity lower than 106 mPa·s at ambient temperature; the first photopolymerizable functional group and the second photopolymerizable functional group comprises a —C≡C— bond and are capable of polymerization and/or crosslinking initiated by light, and upon application of the composition, the oil phase and the aqueous phase are capable of spontaneously forming a form a bilayer and forming a bionic dressing with a two-layer cross-linked structure.

The bionic dressing-forming composition described herein can conveniently form a bionic dressing. When applying, the oil phases and the aqueous phases in the composition can be mixed (for example, by shaking) and then applied (for example, sprayed or coated) to the wound. The oil phase and the aqueous phase in the composition can rapidly and spontaneously separate to form an oil phase-aqueous phase double-layer structure, and a double-layer structure consisting of adjacent skin conforming layers and outer layers can be formed by photoinitiated polymerization/crosslinking curing, wherein the skin conforming layer is formed by the aqueous phase and the outer layer is formed by the oil phase. In certain embodiments, photoinitiated polymerization/crosslinking is performed by reacting the prepolymers or monomers in the oil phase and the aqueous phase by exposure to natural light or ultraviolet light, preferably by exposure to ultraviolet light, after application of the composition.

In certain embodiments, the aqueous phase and the oil phase in the bionic dressing-forming composition may each have an intrinsic viscosity of less than about 1000 mPa·s, 100 mPa·s, 90 mPa·s, 80 mPa·s, 70 mPa·s, 65 mPa·s, 60 mPa·s, 55 mPa·s, 50 mPa·s, 45 mPa·s, 40 mPa·s, 35 mPa·s, 30 mPa·s, 25 mPa·s, 20 mPa·s, 15 mPa·s, 10 mPa·s, 9 mPa·s, 8 mPa·s, 5 mPa·s, 4 mPa·s, 3 mPa·s or 2 mPa·s, or close to 1 mPa·s at ambient temperature.

In certain embodiments, the aqueous phase and the oil phase in the bionic dressing-forming composition may each have an intrinsic viscosity of less than 1-1000 mPa·s, 1-100 mPa·s, 50-90 mPa·s, 40-80 mPa·s, 30-70 mPa·s, 25-65 mPa·s, 20-60 mPa·s, 15-55 mPa·s, 10-50 mPa·s, 5-45 mPa·s, 4-40 mPa·s, 3-35 mPa·s, 2-30 mPa·s, 1-25 mPa·s or 1-20 mPa·s at ambient temperature.

In the bionic dressing-forming composition described herein, the hydrophobic prepolymer or the hydrophobic monomer included in the oil phase can be a biocompatible hydrophobic prepolymer or monomer, which can be polymerized or crosslinked by photoinitiation to form a hydrophobic polymer. The hydrophilic prepolymer or the hydrophilic monomer included in the aqueous phase can be a biocompatible hydrophilic prepolymer or monomer, which can be polymerized or crosslinked by photoinitiation to form a hydrophilic hydrogel polymer.

In certain embodiments, the first photopolymerizable functional group is one or more of acrylate, methacrylate, 1,6-hexanediol diacrylate, or beta-hydroxyethyl methacrylate.

In certain embodiments, the hydrophobic prepolymer or the hydrophobic monomer also has a hydrophobic group selected from one or more of the following: C10 to C20 hydrocarbon group, ether, amine group, amide group, etc., wherein the C10 to C20 hydrocarbon group may have a single bond, a double bond, or a triple bond.

In certain embodiments, the hydrophobic prepolymer or the hydrophobic monomer itself can serve as the solvent in the oil phase without the need to additionally add other organic solvents. In certain embodiments, the overall viscosity of the oil phase can also be adjusted by adding other organic solvents. Organic solvents that can be used include, but are not limited to absolute ethanol, dichloromethane, etc.

Examples of hydrophobic prepolymers or hydrophobic monomers useful in the bionic dressing-forming composition described herein may be selected from one or more of poly (lactic acid-propylene glycol-lactic acid) dimethyl acrylate, polypropylene glycol diacrylate, polypropylene glycol dimethacrylate, polypropylene acrylate, and polymethyl methacrylate.

The oil phase and/or the aqueous phase can also comprise other functional material components, such as antimicrobial active ingredients. In certain embodiments, the antimicrobial active ingredients included in the oil phase are triclosan (TCS) and/or curcumin. Since the oil phase has a slower degradation rate than the aqueous phase, the above-mentioned antimicrobial active ingredients can be included in the oil phase to facilitate long-lasting antimicrobial activity.

For example, when triclosan is added to the oil phase, the antibacterial effect of the bionic dressing described herein can be further promoted to facilitate rapid wound healing. Curcumin can reduce inflammation by regulating the expression of inflammation-related factors and cytokines, reduce oxidative stress by neutralizing free radicals, and also has antimicrobial activity against most bacteria and fungi, and can promote the regeneration of blood vessels and tissues, promote the proliferation of fibroblasts, and improve the blood supply of wound areas.

In certain embodiments, the aqueous phase of the bionic dressing-forming composition also comprises antimicrobial active ingredients, such as triclosan (TCS) and/or curcumin, etc.

In certain embodiments, the concentration of the antimicrobial active ingredient in the oil phase is 0-20 wt/wt %, 1-10 wt/wt %, 2-7 wt/wt %, 3-8 wt/wt % or 5 wt/wt %. For example, the concentration of the antimicrobial active ingredient in the oil phase is 1 wt/wt %, 1.5 wt/wt %, 2 wt/wt %, 2.5 wt/wt %, 3 wt/wt %, 3.5 wt/wt %, 4 wt/w %, 4.5 wt/wt %, 5 wt/wt %, 5.5 wt/wt %, 6 wt/wt %, 7 wt/wt %, 8 wt/wt %, 9 wt/wt %, 10 wt/wt %, 15 wt/wt % or 20 wt/wt %, or any content range therebetween.

The oil phase and/or the aqueous phase of the bionic dressing-forming composition also comprises a first photoinitiator for initiating polymerization and/or crosslinking of hydrophobic prepolymers or hydrophobic monomers or a second photoinitiator for initiating polymerization and/or crosslinking of hydrophilic hydrogel prepolymer or hydrophilic hydrogel monomers. The first/second photoinitiator that can be used in the present invention can be: quinolones, such as 2-hydroxy-1-[4-(2-hydroxyethoxy)phenyl]-2-methyl-1-propanone, di (2,4,6-trimethylbenzoyl)phenylphosphine oxide, benzylquinolone, camphorquinone (CQ); organic dyes, such as Rhodamine B, phthalocyanine, eosin Y (EY); metal complexes, such as lithium phenyl-2,4,6-trimethylbenzoylphosphinate (LAP), copper (II) phthalocyanine (copper phthalocyanine), ruthenium pyridine complex Ru(II)(bpy)3]2+/sodium sulfate (SPS); dibenzothiazole derivatives, such as 2,4,6-triphenyldibenzothiazole (TPD), 2,6-dimethylbenzothiazole; or phenolic derivatives, such as phenol-methoxyacrylate or hydroxybenzoate. In certain embodiments, specific examples of the first photoinitiator for initiating polymerization and/or crosslinking of the hydrophobic prepolymer or the hydrophobic monomer included in the oil phase of the bionic dressing-forming composition may include di (2,4,6-trimethylbenzoyl)phenylphosphine oxide, lithium phenyl-2,4,6-trimethylbenzoylphosphinate (LAP), etc. Examples of the second photoinitiator included in the composition for initiating polymerization and/or crosslinking of the hydrophilic hydrogel prepolymer or the hydrophilic hydrogel monomer include 2-hydroxy-1-[4-(2-hydroxyethoxy)phenyl]-2-methyl-1-propanone or camphorquinone (CQ).

In certain embodiments, the second photopolymerizable functional group is acrylic acid, acrylamide, methacrylamide, etc.

In certain embodiments, the hydrophilic prepolymer or the hydrophilic monomer also has a hydrophilic group selected from one or more of the following: thiol, carboxyl and hydroxyl groups.

In certain embodiments, the concentration of the hydrophilic prepolymer or the hydrophilic monomer in the aqueous phase may be 1-30 wt/wt %. In certain embodiments, the concentration of the hydrophilic prepolymer or the hydrophilic monomer in the aqueous phase can be 3 wt/wt %, 5 wt/wt %, 7 wt/wt %, 9 wt/wt %, 11 wt/wt %, 13 wt/wt %, 15 wt/wt %, 17 wt/wt %, 19 wt/wt %, 20 wt/wt %, 21 wt/wt %, 23 wt/wt %, 25 wt/wt %, 27 wt/wt %, 29 wt/wt %, 30 wt/wt %, or any numerical range therebetween. In certain embodiments, the concentration of the hydrophilic prepolymer or the hydrophilic monomer in the aqueous phase is 5-20 wt/wt %. The viscosity of the aqueous phase can be adjusted by adjusting the concentration of the hydrophilic prepolymer or the hydrophilic monomer in the aqueous phase or adjusting the water content in the aqueous phase.

Examples of hydrophilic hydrogel prepolymers or hydrophilic hydrogel monomers useful in the bionic dressing-forming composition described herein include, but are not limited to, one or more of methacrylated gelatin, acrylated gelatin, methacrylated hyaluronic acid, acrylated hyaluronic acid, methacrylated chitosan, acrylated chitosan and hydrophilic polyethylene glycol diacrylate.

In certain embodiments, the aqueous phase of the bionic dressing-forming composition may also comprise an active ingredient that is soluble therein. The active ingredient may be a hemostatic active ingredient and/or a wound healing active ingredient. The hemostatic active ingredient may be, for example, a water-soluble salt or thrombin, etc., and the wound healing active ingredient may be, for example, a growth factor, etc., and the precise medical needs of different wound conditions are met. The water-soluble salt may be, for example, calcium ions (such as CaCl₂). The growth factor may be, for example, vascular endothelial growth factor.

When calcium ions are added to the aqueous phase, the bionic dressing described herein can activate the coagulation cascade reaction, thereby further promoting rapid hemostasis. In certain embodiments, the concentration of the soluble calcium salt (Ca²⁺) in the aqueous phase is 0-70 wt/wt %. For example, the concentration of the soluble calcium salt (Ca²⁺) in the aqueous phase is 0.1 wt/wt %, 0.5 wt/wt %, 1 wt/wt %, 3 wt/wt %, 5 wt/wt %, 8 wt/wt %, 10 wt/wt %, 11 wt/wt %, 12 wt/wt %, 15 wt/wt %, 20 wt/wt %, 25 wt/wt %, 30 wt/wt %, 35 wt/wt %, 40 wt/wt %, 50 wt/wt %, 60 wt/wt % or 70 wt/wt % or any content range therebetween, such as 5-30 wt/wt %, 5-20 wt/wt % or 5-10 wt/wt %.

The inventors found that by adjusting the concentration of the calcium salt to the above range, it is also possible to make the mixture formed to maintain the oil/aqueous phase mixture within a desired time period (for example, within 5 seconds, within 10 seconds, within 15 seconds or within 20 seconds or a longer period of time) before light curing for easier application by the user.

In the above embodiments of the present invention, the amount of photoinitiator in the oil phase or the aqueous phase should be sufficient to initiate the polymerization or crosslinking reaction of the prepolymer or the monomer therein, for example, the amount of the first photoinitiator in the oil phase can be 0.1-5 wt/wt %, such as 0.5 wt/wt %, 1 wt/wt %, 1.5 wt/wt %, 2 wt/wt %, 2.5 wt/wt %, 3 wt/wt %, 3.5 wt/wt %, 4 wt/wt %, 4.5 wt/wt %, 5 wt/wt % or any numerical range therebetween; and/or the amount of the second photoinitiator in the aqueous phase can be 0.05-3 wt/wt %, such as 0.1 wt/wt %, 0.25 wt/wt %, 0.3 wt/wt %, 0.4 wt/wt %, 0.5 wt/wt %, 1 wt/wt %, 1.5 wt/wt %, 2 wt/wt %, 2.5 wt/wt %, 3 wt/wt % or any numerical range therebetween. The crosslinking degree of the hydrophobic polymer and/or the hydrophilic hydrogel obtained after crosslinking can be, for example, at least 50%, 60%, 65%, 70%, 75%, 80%, 85%, 90%, 95% or 100%.

In certain embodiments, the volume ratio of the oil phase and the aqueous phase of the bionic dressing-forming composition is from 3:1 to 1:3, 2:1, or 1:1.

In certain embodiments, the bionic dressing-forming composition also includes additional additives in the oil phase and/or the aqueous phase to achieve additional functions of the dressing. The additives may be, for example, one or more of the following: antimicrobial agents, odor control agents, colorants, wound pH, and blood glucose monitoring agents.

In certain embodiments, at the interface between the skin conforming layer and the outer layer, there is a cross-link between the hydrophilic hydrogel and the hydrophobic polymer formed after the crosslinking and curing, such that the interface between the skin conforming layer and the outer layer has strong adhesion force to avoid interlayer peeling.

In certain embodiments, the oil phase of the bionic dressing-forming composition described herein comprises poly (lactic acid-propylene glycol-lactic acid) dimethacrylate (abbreviated as PGLADMA or PLD) and triclosan, and the aqueous phase comprises gelatin methacryloyl (abbreviated as GelMA) and calcium salt, and a photoinitiator for initiating photopolymerization may be included in the oil phase and the aqueous phase. Due to the acrylic or acryloyl functional groups present in PGLADMA and GelMA, the bionic dressing-forming composition is able to cure via photo-crosslinking within 100 seconds and achieves strong adhesion force at the interface between the skin conforming layer and the outer layer to avoid interlayer peeling. In addition, due to the tissue adhesion of GelMA and the elasticity of GelMA and PGLADMA, the bionic dressing formed after curing can firmly adhere to the wound site and adapt to joint movements. In addition, the relatively soft and hydrophilic calcium salt (Ca²⁺) carrier GelMA can activate the coagulation cascade reaction and achieve rapid hemostasis. At the same time, the relatively hard and hydrophobic PGLADMA layer loaded with triclosan (TCS) can act as a protective scab to maintain a moist and sterile environment. These properties work synergistically to enable the dressing described herein to inhibit inflammatory pathways (such as TNF-α), increase M2 macrophage polarization, promote the transition from inflammation to proliferation, and can effectively promote vascular reconstruction by jointly activating and coordinating cGMP/PKG-Wnt/Ca²⁺ signaling pathway, especially beneficial to realize scar-free healing.

The bionic dressing-forming composition described herein can be stored in the dark before use, thereby minimizing the premature reaction of the prepolymer or monomer in the oil phase and the aqueous phase, so as not to affect the use of the composition.

In certain embodiments, the composition does not include any emulsifiers or surfactants so as to facilitate rapid separation of the oil phase and the aqueous phase and reduce the difficulty of subsequent cleaning of the wound and the risk of infection. For example, in certain embodiments, Span 80 or Tween 20 is not included in the composition.

In addition, the bionic dressing-forming composition described herein can be formed into various dosage forms, usually a liquid formulation, such as a spray or other forms of liquid formulation.

When the bionic dressing is formed from the bionic dressing-forming composition, the oil phase and the aqueous phase are uniformly mixed and then applied to the upwardly facing wound surface. Due to the incompatible nature of the oil phase and the aqueous phase, the composition will quickly separate after application. Under the action of gravity, the aqueous phase with high-density is deposited in the lower layer and can adhere to the wound surface due to its good affinity with the skin; the oil phase with low density is located on the layer above the aqueous phase, and the polymers in the two phases are polymerized or cross-linked under the initiation of ultraviolet light or natural light to form the bionic dressing of the present invention. It should be understood that the bionic dressing-forming composition of the present invention can be applied in a variety of ways, such as by spraying, coating or extrusion.

Generally, the bionic dressing-forming composition described herein is cured within 100 seconds, within 80 seconds, within 60 seconds, within 50 seconds, within 40 seconds, within 30 seconds, within 20 seconds, within 10 seconds or less than 10 seconds after photoinitiation.

The curing time can be adjusted by adjusting the concentration of the photoinitiator to the range as described above. For example, the concentration of photoinitiator can be increased to shorten the curing time. For example, after adjusting the concentration of the photoinitiator in the oil phase and the aqueous phase from 1 wt % to 2 wt %, the curing time can be shortened to within 30 seconds.

Specific examples are described below with reference to the accompanying drawings. It should be understood that various modifications are possible without departing from the scope of the invention as described above. The following examples are provided for illustration only.

### EXAMPLES

### Materials

| Material | Source | Description |
|---|---|---|
| Poly(lactic acid-propylene glycol- | Laboratory synthesis | The raw materials used to prepare |
| lactic acid) dimethacrylate | | PGLADMA are lactide, propylene |
| (PGLADMA or PLD) | | glycol, etc. from MACKLIN |
| Gelatin methacryloyl (GelMA) | Laboratory synthesis | |
| Calcium chloride | Commercially available | |
| | products, e.g. purchased from | |
| | Commercially available | |
| | products, e.g. purchased from | |
| | MACKLIN | |
| Triclosan | Commercially available | |
| | products, e.g. purchased from | |
| | MACKLIN | |
| Irgacure2959 ™ | Commercially available | 2-hydroxy-1-[4-(2- |
| | products, e.g. purchased from | hydroxyethoxy)phenyl]-2-methyl- |
| | MACKLIN | 1-propanone |
| Irgacure819 ™ | Commercially available | di(2,4,6- |
| | products, e.g. purchased from | trimethylbenzoyl)phenylphosphine |
| | MACKLIN | oxide |

In the following examples, the intrinsic viscosity of the oil phase or the aqueous phase was tested by a rheometer under rotation mode conditions at ambient temperature.

### Preparation of Bionic Dressing-Forming Compositions

### Example 1

In this example, a bionic dressing-forming composition containing PGLADMA and GelMA as prepolymers was prepared and prepared as a spray or a liquid formulation for coating. The specific preparation steps were as follows: First, 1 wt % of the first photoinitiator Irgacure819™ was dissolved in the liquid PGLADMA as a prepolymer, and 1 wt % second photoinitiator Irgacure2959™ was dissolved in 10 wt % GelMA aqueous solution. The viscosity of the oil phase was measured to be 200 mPa·S, and the viscosity of the aqueous phase was 100 mPa·S. After the photoinitiator was completely dissolved, the oil phase and the aqueous phase were mixed into a spray bottle in a 1:1 ratio and keep away from light until use.

### Example 2

In this example, a bionic dressing-forming composition containing PGLADMA and GelMA as prepolymers was prepared and prepared as a spray or a liquid formulation for coating. The aqueous phase as the lower layer also contains calcium ions Ca²⁺, and the oil phase as the upper layer also contains triclosan. The specific preparation steps were as follows: First, 1 wt % of the first photoinitiator Irgacure819™ was dissolved in the liquid PGLADMA as a prepolymer, and 1 wt % second photoinitiator Irgacure2959™ was dissolved in 10 wt % GelMA aqueous solution. After the photoinitiator was completely dissolved, 5 wt % triclosan was added to the upper oil phase and 10 wt % calcium chloride was added to the lower aqueous phase to prepare a bionic dressing BDM. The viscosity of the oil phase of the prepared bionic dressing BDM was measured to be 200 mPa. S, and the viscosity of the aqueous phase was 100 mPa. S. After thorough stirring and dissolution, the oil phase and the aqueous phase were mixed into a spray bottle in a 1:1 ratio and keep away from light until use.

### Example 2A and Examples 3-7

In these examples, the bionic dressing-forming composition was prepared using the same method as Example 2, except that the concentrations of triclosan and calcium chloride were adjusted.

The concentrations of triclosan and calcium chloride in examples 1-7 are listed in Table 1 below.

TABLE 1

| | Example | Example | Example | Example | Example | Example | Example | Example |
|---|---|---|---|---|---|---|---|---|
| | 1 | 2 | 2A | 3 | 4 | 5 | 6 | 7 |
| Calcium | 0 | 10 | 10 | 5 | 20 | 10 | 10 | 10 |
| chloride | | | | | | | | |
| (wt %) | | | | | | | | |
| Triclosan | 0 | 5 | 0 | 0 | 0 | 2.5 | 5 | 10 |
| (wt %) | | | | | | | | |

### Examples 8-10

In these examples, the bionic dressing-forming composition was prepared using the same method as Example 2, except that the viscosity of the oil phase and the aqueous phase was adjusted, for example, by adjusting the amount of a solvent.

TABLE 2

| | Example | Example | Example |
|---|---|---|---|
| | 8 | 9 | 10 |
| Viscosity of the | 80 | 70 | 90 |
| oil phase | | | |
| mPa · S | | | |
| Viscosity of the | 20 | 15 | 30 |
| aqueous phase | | | |
| mPa · S | | | |

The bionic dressing described herein can be successfully obtained by using the bionic dressing-forming composition in the above preparation examples.

### Forming the Bionic Dressing

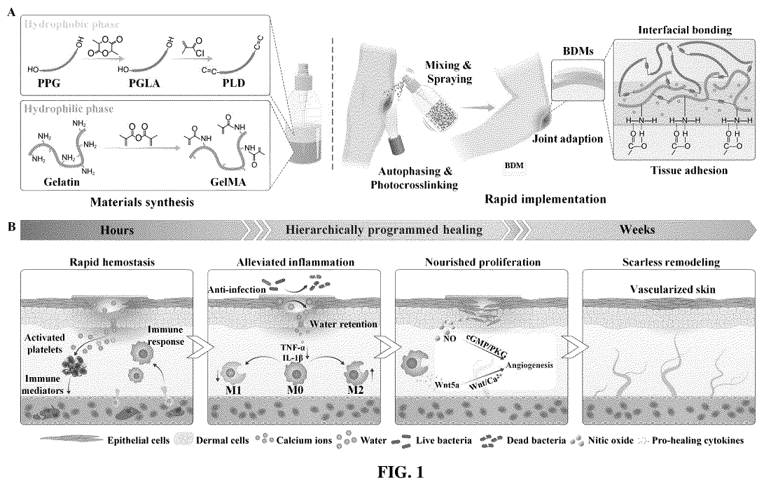

FIG. 1 shows the materials contained in the bionic dressing-forming composition provided in preparation examples 1-10 and the formation process of the bionic dressing. As shown in FIG. 1, in the composition, the oil phase contains PGLADMA, and the aqueous phase contains GelMA. Both PGLADMA and GelMA can be rapidly photo-crosslinked under ultraviolet light irradiation. In the case where the composition was a spray, the composition in the form of a spray was shaken to mix the oil phase and the aqueous phase and then sprayed onto the wound site on skin. In the case where the composition was a liquid formulation for application, the composition was shaken to mix the oil phase and the aqueous phase and then applied to the wound site on skin. Due to the immiscibility of the oil phase and the aqueous phase, the composition will quickly separate after application. Under the action of gravity, the aqueous phase with high-density is deposited in the lower layer and can adhere to the wound surface due to its good affinity with the skin; the oil phase with low density is located on the layer above the aqueous phase, and the polymers in the two phases are polymerized or cross-linked under the initiation of ultraviolet light or natural light to form the bionic dressing described herein.

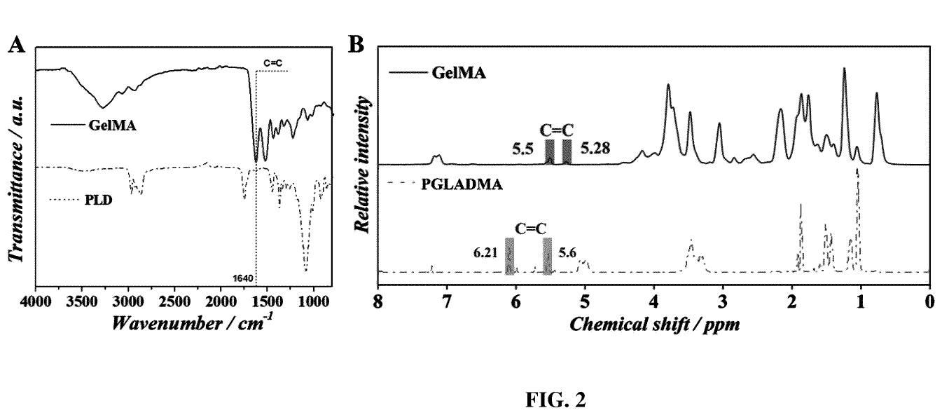

FIGS. 2A-2B show the test results of Fourier transform infrared spectroscopy (FTIR) and ¹H nuclear magnetic resonance spectroscopy (¹H NMR) to verify the chemical structures of GelMA and PGLADMA. In FIG. 2A, the 1640 cm⁻¹ position of the FTIR spectrum reflects the characteristic peak of the C═C double bond of the two materials; In the ¹H NMR results in FIG. 2B, GelMA has characteristic peaks of C═C double bonds at 5.5 ppm and 5.28 ppm, and PGLADMA has characteristic peaks of C═C double bonds at 6.21 and 5.6 ppm.

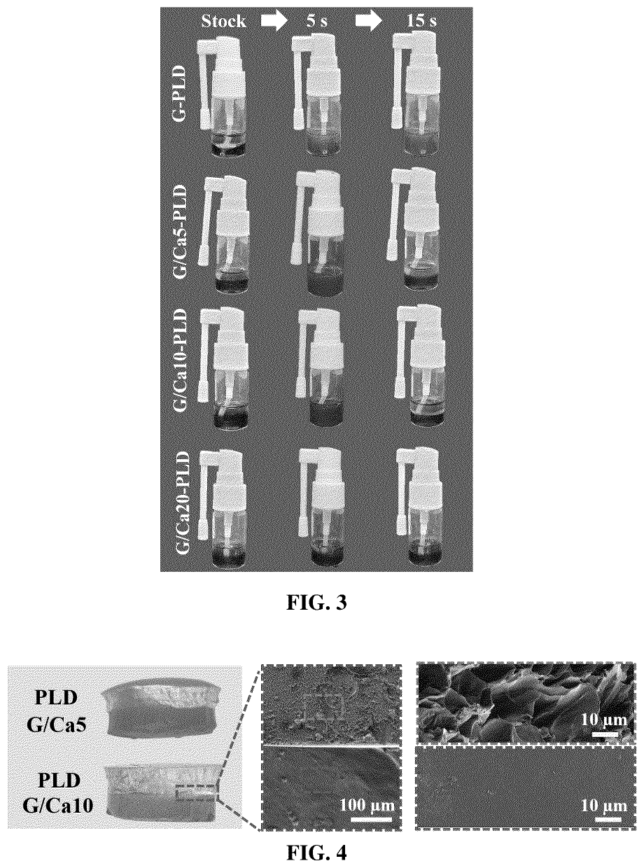

FIG. 3 shows the spontaneous water/oil separation process without the addition of CaCl₂) and with the intervention of different weight fractions of calcium chloride (CaCl₂) (preparation examples 1-4). Each sample contained GelMA and PGLADMA in the aqueous phase and the oil phase respectively. G-PLD (preparation example 1) indicated that CaCl₂) was not added, G/Ca⁵-PLD (preparation example 3), G/Ca¹⁰-PLD (preparation example 2A) and G/Ca²⁰-PLD (preparation example 4) indicated that different concentrations of CaCl₂) (5 wt %, 10 wt %, 20 wt %) were added to the GelMA aqueous solution. As shown in FIG. 3, for samples without CaCl₂) and samples with different concentrations of CaCl₂), GelMA (aqueous phase) and PGLADMA (oil phase) can realize rapid layering after mixing and shaking. For samples with CaCl₂), rapid layering can be realized within 15 s after mixing and shaking. For sample G-PLD without CaCl₂), rapid layering can be realized within a few minutes after mixing and shaking.

FIG. 4 is a macroscopic view of a bionic dressing formed after photocrosslinking and a scanning electron microscope view of a material and a crosslinking interface provided by an example, wherein the bionic dressing is formed by the compositions of preparation example 3 and preparation example 2A, respectively. Both the macroscopic view and the scanning electron SEM results reflect the layered structure of GelMA and PGLADMA after crosslinking.

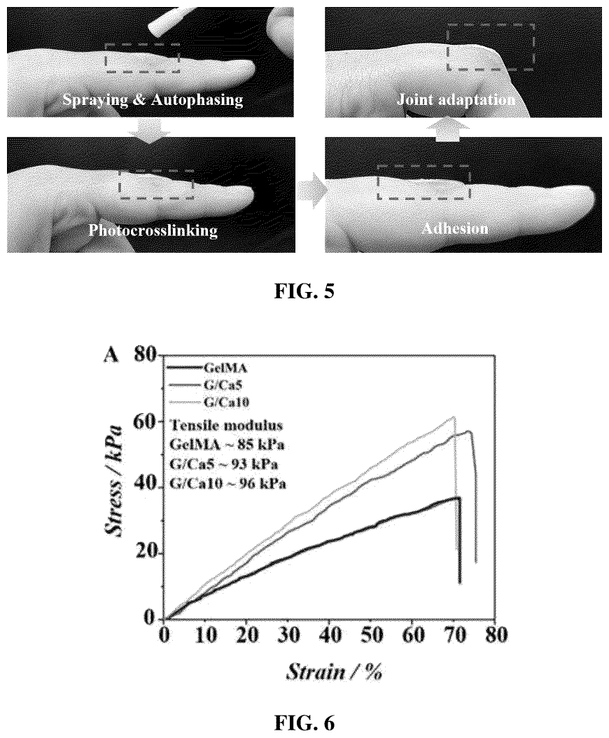

The photos in FIG. 5 illustrate the application of the bionic dressing-forming composition of preparation example 1, and demonstrate the strong tissue adhesion and excellent joint movement adaptability of the formed bionic dressing, indicating that the bionic dressing can adhere closely to tissues and adapt to joint activities. The bionic dressing formed from the bionic dressing-forming composition of examples 2-10 can also achieve the same strong tissue adhesion and excellent joint motion adaptability.

Performance testing of the resulting bionic dressing

In this example, the mechanical properties, hemostatic and microbicidal properties, biocompatibility, etc. of the formed bionic dressing were tested.

The tests in this example were conducted under the following conditions/standards.
5. Mechanical property test: The mechanical properties of bionic excipients were based on the ASTM F2458-05 standard. GelMA, PGLADMA and double-layer GelMA/PGLADMA samples with a size of 20×5 mm were prepared and stretched at a rate of 1 mm/min using a two-arm mechanical tensile instrument at room temperature, and the mechanical properties were calculated, where tensile elastic modulus=stress/strain. For example, reference can be made to the test method in Theocharidis G, Yuk H, Roh H, et al. A strain-programmed patch for the healing of diabetic wounds [J]. Nature biomedical engineering, 2022, 6 (10): 1118-1133.
6. Hemostatic property: For the hemostatic properties of bionic excipients, for example, reference can be made to Guo Y, Wang Y, Zhao X, et al. Snake extract-laden hemostatic bioadhesive gel cross-linked by visible light [J]. Science Advances, 2021, 7 (29): cabf9635.
7. Microbicidel property: For the microbicidel property of bionic excipients, reference can be made to He W, Bai J, Chen X, et al. Reversible dougong structured receptor-ligand recognition for building dynamic extracellular matrix mimics [J]. Proceedings of the National Academy of Sciences, 2022, 119 (8): c2117221119.
8. Biocompatibility: The biocompatibility of bionic excipients can refer to for example, the ISO-10993 standard. All samples (10 mm) were soaked in the complete culture medium at 37° C. for 24 hours, and then cells were inoculated into a 24-well plate at a density of 1×10⁴ cells/cm² using the extracted culture medium. After 1, 2, and 3 days of incubation, cell viability and cell proliferation were evaluated using a live/dead cell detection kit (Thermo Fisher, Hong Kong) and a CCK-8 kit (Sigma-Aldrich, Hong Kong), respectively.

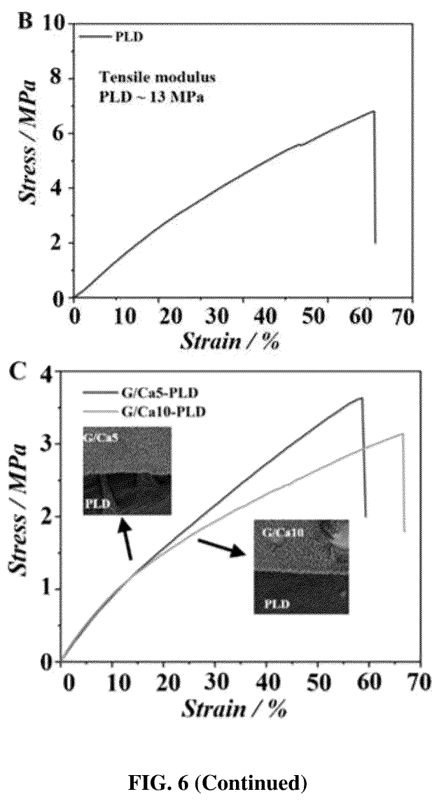

FIG. 6A shows a schematic diagram of the mechanical property test results of the aqueous phase in preparation examples 1-3 after photo-initiated crosslinking and curing. As shown in FIG. 6A, the elastic modulus of the skin conforming layer formed by curing GelMA with different contents of CaCl₂) was approximately 85-96 kPa. FIG. 6B shows a schematic diagram of the mechanical property test results of the oil phase in preparation example 1 after photo-initiated crosslinking and curing. Only the elastic modulus of the cross-linked outer layer formed by PGLADMA after curing was approximately 13 MPa, indicating that the outer layer structure had good tensile properties after crosslinking. FIG. 6C shows a schematic diagram of the mechanical property test results of the compositions in preparation example 2A and preparation example 3 after photo-initiated crosslinking and curing. The elastic modulus was about 4-10 MPa. The inset shows the SEM results of the stretched double-layer structure, indicating that the clear crosslinking interface of the double-layer dressing can still be maintained after stretching

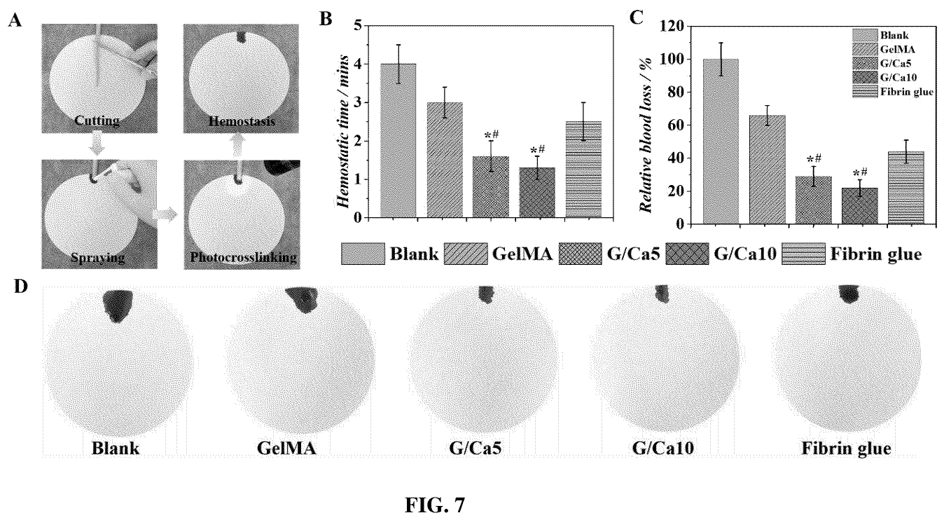

FIG. 7 shows the hemostatic property test results of a double-layer anti-scar dressing provided by the example of the present invention using a rat tail truncated model as an example. The blank control indicated that the rat tail was truncated, and bleeding was stopped by the own coagulation mechanism alone without any treatment. Sample GelMA indicated that calcium chloride was not added to the aqueous phase, and samples G/Ca⁵ and G/Ca¹⁰ indicated that calcium chloride was added to the aqueous phase at 5 wt % and 10 wt %, respectively. As shown in the photo in FIG. 7A, after 30% of the rat's tail was truncated during the experiment, the rat's tail would bleed heavily. The dressing-forming composition in this embodiment can achieve rapid hemostasis. As shown in FIGS. 7B-7D, compared to the blank control, the dressings formed from the compositions of preparation examples 1-3 of the present invention can achieve effective hemostasis. In the case of containing calcium ions, compared to sample GelMA, both samples G/Ca5 and G/Ca10 can achieve shorter hemostasis time and less blood loss, among which when containing 10 wt % CaCl₂, the best hemostatic effect was achieved, achieving rapid hemostasis within 90 seconds.

FIG. 8 shows the evaluation results of the microbicidal ability of the dressing formed by the composition (preparation examples 5-7) containing PGLADMA and triclosan (TCS) in the oil phase, taking E. coli and S. aureus as examples.

FIG. 8A shows the inhibition zone experiment, in which the larger the transparent circle, the better the antimicrobial effect. As shown in the figure, the dressing containing TCS in the oil phase showed better antimicrobial effect, and as the TCS content increased to 10 wt %, the antimicrobial effect became more significant. FIG. 8B shows the quantitative results of the inhibition zone experiment, which was calculated based on the area of the transparent circle in FIG. 8A. As the TCS content increased, the antimicrobial effect became more significant.

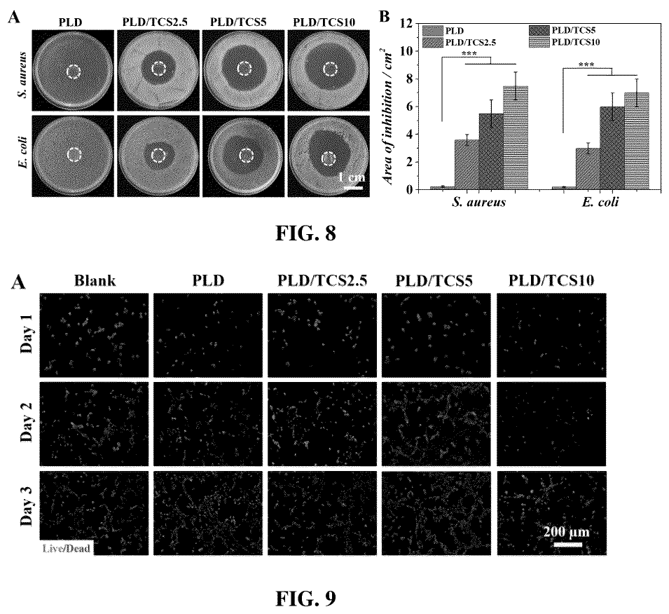

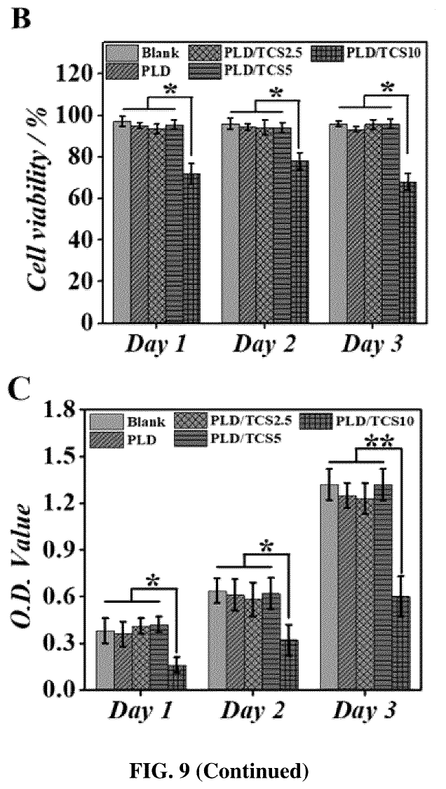

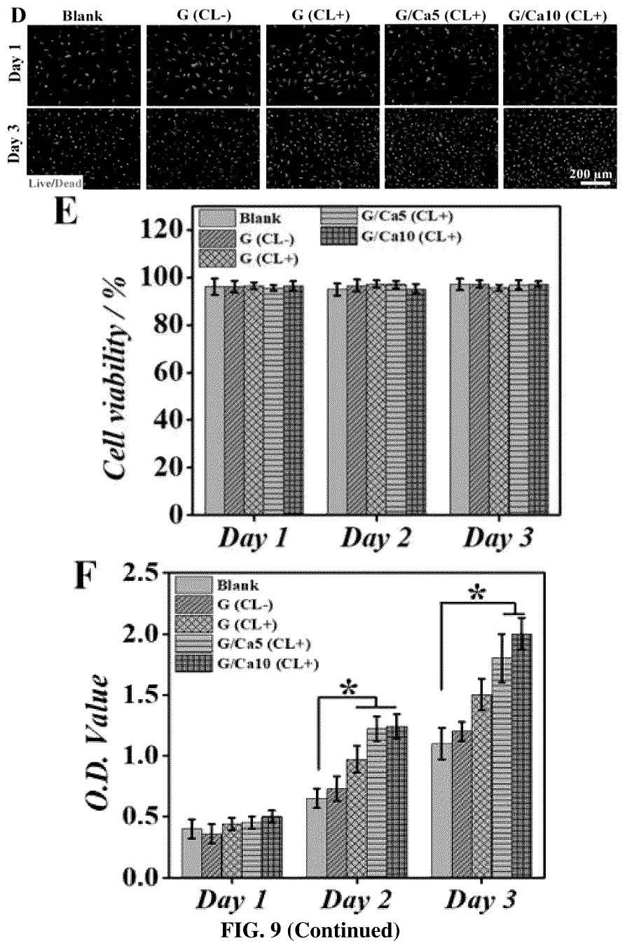

FIGS. 9A to 9C show the biocompatibility evaluation results of the dressings formed from the compositions of preparation examples 1 and 5-7. FIG. 9A shows the effect of mixing different TCS contents on the compatibility of 3T3 cells. The cells represented by gray highlights are viable cells. The results in FIG. 9A show that TCS has no toxicity to cells at a concentration below 5%, and is only slightly toxic at a concentration of around 10%. FIG. 9B shows the quantitative results of cell viability, and more than 95% represents good compatibility. As shown in FIG. 9B, the dressings formed by the compositions of preparation examples 1 and 5-7 of the present invention have achieved good compatibility, and the cell viability is above 95%. FIG. 9C shows a cell proliferation experiment, and the O.D. value indicates the number of cells. FIG. 9C shows that the dressings formed by the compositions of preparation examples 1 and 5-7 of the present invention all show a significant increase in the number of cells.

The results of FIG. 9D to FIG. 9F show that the addition of collagenase (CL+means the addition of collagenase, CL-means no addition of collagenase) is beneficial to further enhancing the antimicrobial effect of the dressing of the present invention, and the obtained dressing still has good biocompatibility.

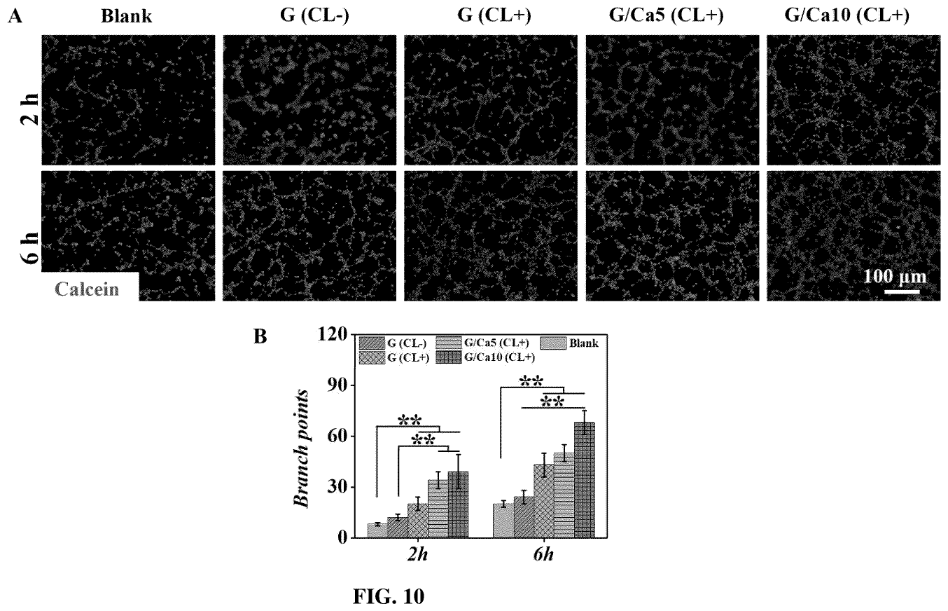

FIGS. 10A-10B show the results of evaluating the in vitro angiogenesis ability of the dressing after the aqueous phases (GelMA+calcium chloride solution) in the compositions of preparation examples 1-3 were cured, taking human umbilical vein endothelial cells (HUVEC) as an example. FIG. 10A shows the experiment results of the angiogenesis ability of cells, indicating for the angiogenesis ability of endothelial cells, the denser the network and the more nodes, the better the angiogenesis ability. FIG. 10B shows the quantification of angiogenesis. The ordinate shows the number of cross-linked nodes of the vascular network calculated by Image J software. As shown in FIGS. 10A and 10B, the dressings formed by the present invention all have strong angiogenesis ability, especially when containing collagenase and calcium ions, they have improved angiogenesis ability (up to more than 60 nodes can be obtained in 6 hours).

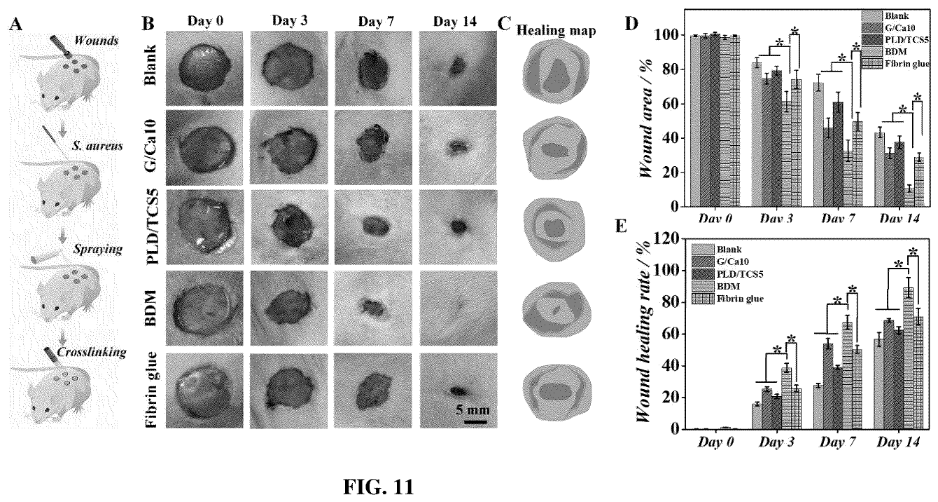

In FIGS. 11A-11C, taking the full-thickness skin wound model of rats infected by Staphylococcus aureus as an example, the BDM formed by the composition of preparation example 2 of the present invention, and the bionic dressing formed by PLD/TCS5 monolayer (formed by curing the oil phase containing PLD and 5 wt % TCS) and G/Ca¹⁰ monolayer (formed by curing the aqueous phase containing GelMA and 10 wt % CaCl₂) were evaluated for the treated wound healing effect based on the area change and healing ratio of the wound. The photos in FIGS. 11A-11C illustrate the rat skin infection defect model and rat wound repair. As shown in the figure, the dressing of the present invention has strong wound repair ability. In particular, the dressing containing TCS can achieve significantly better repair effects than the commercially available control product Fibrin glue. The quantitative results in FIGS. 11D-11F also show this trend. In particular, BDM treatment achieved the most excellent wound healing effect. At 14 days, the wound area treated by BDM was almost invisible, and no scars were found. Correspondingly, the wound repair rate was as high as about 99-100%.

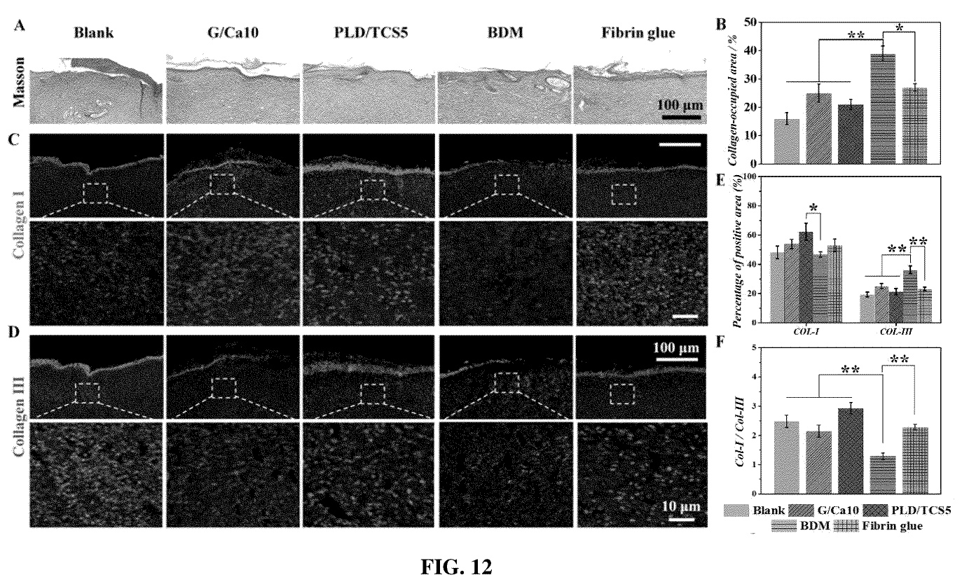

FIG. 12 shows that the bionic dressing (BDM) formed by the composition of preparation example 2 of the present invention and the bionic dressing formed by PLD/TCS5 monolayer (formed by curing the oil phase containing PLD and 5 wt % TCS) and G/Ca¹⁰ monolayer (formed by curing the aqueous phase containing GelMA and 10 wt % CaCl₂) were evaluated for the anti-scar effect by Masson staining of rat skin tissue and immunofluorescence staining of collagen.

FIG. 12A shows the results of Masson staining. The more lighter gray parts, the better the wound repair. As shown in the figure, BDM shows the largest area of lighter gray parts, indicating its best wound repair effect.

The quantitative results of Masson staining shown in FIG. 12B show that compared to other control groups, the BDM group has significantly more new collagen deposition (about 40%) and better skin repair effect.

FIG. 12C and FIG. 12D show the results of immunofluorescence staining for type I and type III collagen. The results show that type I collagen (lighter gray area) is produced less and type III collagen (dark area) is produced more in the BDM group.

FIG. 12E shows the results of quantification of collagen staining area. The results showed that the positive area of type I collagen in the BDM group was lower than that of other control groups, while the positive area of type III collagen (about 40%) was significantly higher than that of other control groups.

FIG. 12F shows the quantification of the ratio of type I and type III collagen. The lower the value, the better the repair and the fewer the scars. As shown in the figure, the BDM group has the lowest quantitative ratio of type I and type III collagen, indicating that compared to the control group, the BDM group according to the present invention has a better wound repair effect and produces fewer scars.

BDMs enhanced wound healing in full-thickness porcine skin wound model.

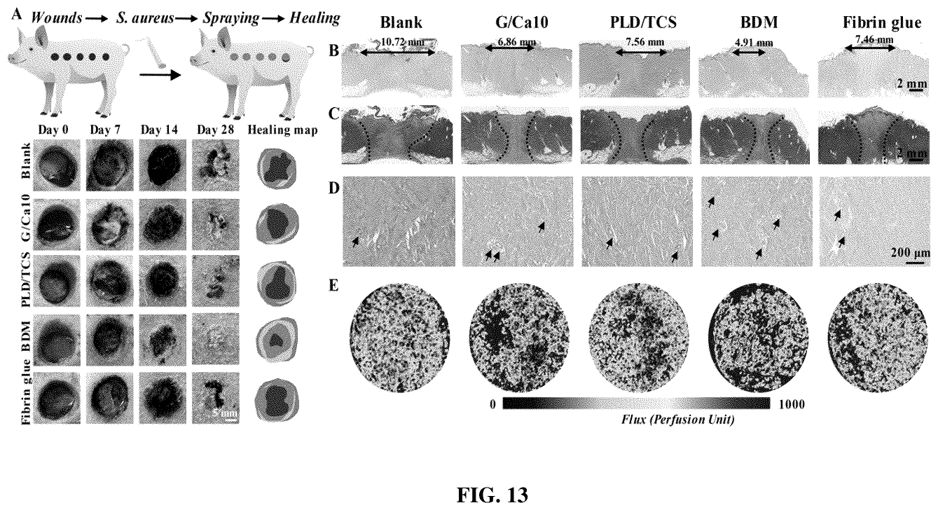

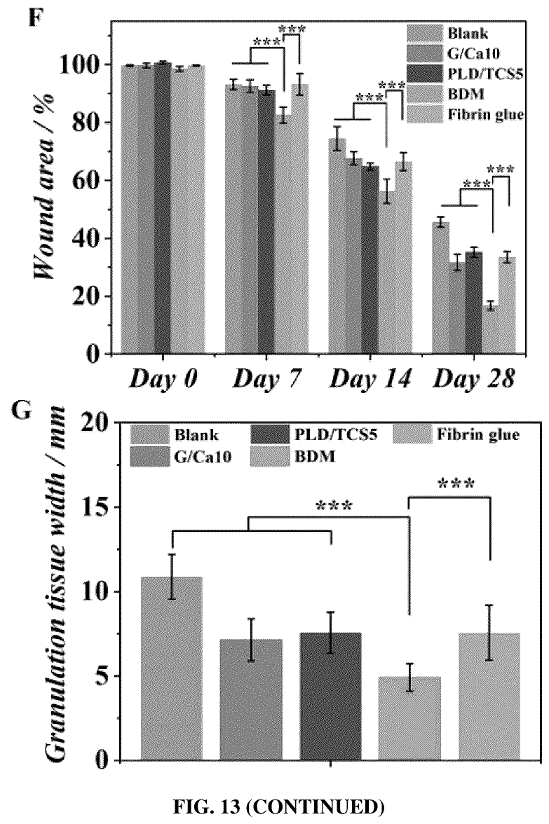

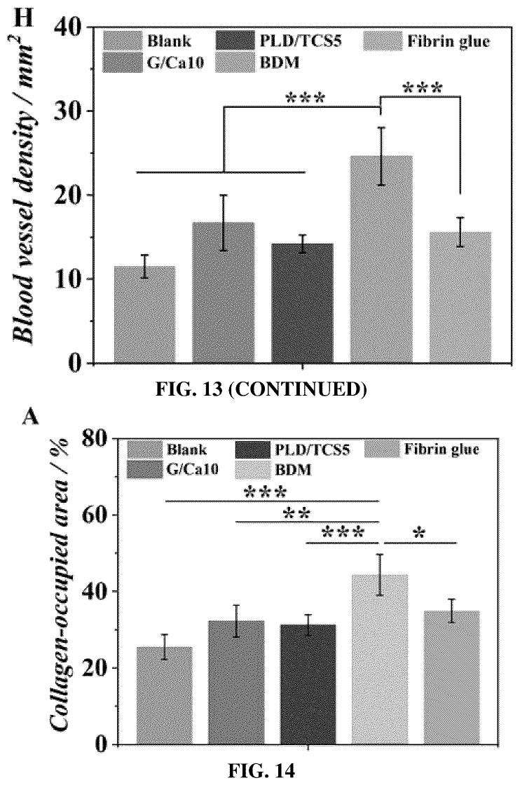

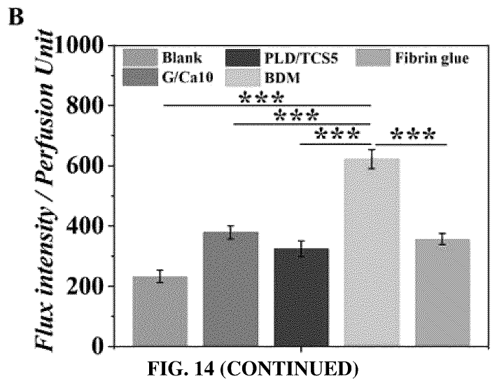

To provide more insight into the therapeutic wound healing efficacy of our BDMs with closer human skin resemblance, we further performed the full-thickness porcine wound model, which is widely recognized for its physiological and anatomical similarities to human skin. We found our BDM substantially accelerated wound closure throughout the healing process. By day 7, the release of TCS from the BDMs significantly reduced the infection on the wound sites, decreasing the inflammation reaction (FIG. 13A). Additionally, the wound area of the BDMs group decreased to around 83.6%, compared to 95.1% in the blank group and 91.2% in the fibrin glue group. By day 28, wound closure in the BDMs group was nearly complete, whereas 50% of the wound in the control group remained unhealed. Moreover, histological H&E staining analysis showed that the width of the granulation tissue in the BDMs group was significantly narrower than that in other groups, indicating minimal scar hyperplasia in the newly formed tissue (FIGS. 13B and G). Furthermore, Masson's trichrome staining revealed the most extensive collagen deposition in the BDM group compared to other groups, demonstrating the effectiveness of BDMs to facilitate the reconstruction of collagen during wound healing (FIG. 13C and FIG. 14A). To further analyze the formation and functionality of the newly formed blood vessels, we quantified red blood cell perfusion with luminal structure formation using H&E staining (FIG. 13D). We discovered that the BDMs group had a higher number of newly formed vessel lumen structures, 2.13 folds and 1.57 folds compared to control and fibrin glue groups respectively, highlighting its superior angiogenic capability (FIG. 13H). Moreover, to quantitatively assess blood flow within the healing tissue, we conducted laser Doppler perfusion imaging (FIG. 13E). We found that the BDMs group exhibited highest blood flow flux with 1.74 folds higher to fibrin glue, indicating the reconstruction of functional blood flow (FIG. 14B). These results collectively demonstrated that our BDMs not only promote the formation of endothelial cell structures but also enhance the development of fully functional, perfused vascular networks, which are crucial for successful wound healing.

The above description of the examples is to facilitate those of ordinary skill in the art to understand and apply the present invention. It is obvious that those skilled in the art can easily make various modifications to these examples and apply the general principles described herein to other examples without inventive efforts. Therefore, the present invention is not limited to the specific examples disclosed herein. Improvements and modifications made by those skilled in the art based on the principles of the present invention without departing from the scope of the present invention should be within the protection scope of the present invention.

What is claimed is:

1. A bionic dressing-forming composition comprising an aqueous phase and an oil phase, wherein the oil phase comprises a hydrophobic prepolymer or a hydrophobic monomer, wherein the hydrophobic prepolymer or the hydrophobic monomer has a first photopolymerizable functional group, and the oil phase has an intrinsic viscosity lower than 106 mPa·s at ambient temperature;
the aqueous phase comprises a hydrophilic hydrogel prepolymer or a hydrophilic hydrogel monomer, and an active ingredient, wherein the hydrophilic hydrogel prepolymer or the hydrophilic hydrogel monomer has a second photopolymerizable functional group and the aqueous phase has an intrinsic viscosity lower than 106 mPa·s at ambient temperature;
the first photopolymerizable functional group and the second photopolymerizable functional group comprises a —C═C— bond and are capable of photopolymerization, and
upon application of the composition, the oil phase and the aqueous phase are capable of forming a bilayer thereby forming a bionic dressing with a double-layer cross-linked structure comprising the hydrophobic polymer and hydrophilic hydrogel.

2. The bionic dressing-forming composition of claim 1, wherein the first photopolymerizable functional group and the second photopolymerizable functional group comprise one or more of acrylate, methacrylate, acrylamide, methacrylamide, styrene, N-vinylpyrrolidone, hydroxybutyl vinyl ether, diethylene glycol divinyl ether, or phenyl glycidyl ether.

3. The bionic dressing-forming composition of claim 1, wherein the hydrophobic prepolymer or the hydrophobic monomer comprises one or more of poly (lactic acid-propylene glycol-lactic acid) dimethyl acrylate, polypropylene glycol diacrylate, polypropylene glycol dimethacrylate, polypropylene acrylate, and polymethyl methacrylate.

4. The bionic dressing-forming composition of claim 1, wherein the composition further comprises a first photoinitiator for initiating polymerization reaction of the hydrophobic prepolymer or the hydrophobic monomer.

5. The bionic dressing-forming composition of claim 1, wherein the hydrophilic hydrogel prepolymer or the hydrophilic hydrogel monomer comprises one or more of methacrylated gelatin, acrylated gelatin, methacrylated hyaluronic acid, acrylated hyaluronic acid, methacrylated chitosan, acrylated chitosan, and hydrophilic polyethylene glycol diacrylate.

6. The bionic dressing-forming composition of claim 1, wherein the composition further comprises a second photoinitiator for initiating photopolymerization reaction of the hydrophilic hydrogel prepolymer or the hydrophilic hydrogel monomer.

7. The bionic dressing-forming composition of claim 1, wherein the active ingredient in the aqueous phase is selected from a hemostatic active ingredient, a wound healing active ingredient, and a combination thereof.

8. The bionic dressing-forming composition of claim 1, wherein the concentration of the active ingredient in the aqueous phase is 0.1-70 wt/wt %

9. The bionic dressing-forming composition of claim 1, wherein at least one of the oil phase and the aqueous phase further comprises an antimicrobial active ingredient.

10. The bionic dressing-forming composition of claim 9, wherein the antimicrobial active ingredient is selected from triclosan and curcumin.

11. The bionic dressing-forming composition of claim 1, wherein the concentration of the antimicrobial active ingredient in the oil phase is 0.1-20 wt %.

12. The bionic dressing-forming composition of claim 1, wherein the composition does not comprise an emulsifier or a surfactant.

13. The bionic dressing-forming composition of claim 1, wherein the composition forms a bionic dressing with a double-layer cross-linked structure within 100 seconds after photoinitiation.

14. The bionic dressing-forming composition of claim 1, wherein the composition is in the form of a liquid formulation.

15. The bionic dressing-forming composition of claim 14, wherein the liquid formulation is in the form of a spray.

16. The bionic dressing-forming composition of any one of claim 1, wherein the composition comprises:
an oil phase comprising poly (lactic acid-propylene glycol-lactic acid) dimethacrylate and triclosan; and
an aqueous phase comprising gelatin methacryloyl and a water-soluble calcium salt.

17. A bionic dressing formed from the bionic dressing-forming composition of claim 1, wherein the bionic dressing comprises at least one double-layer structure having a skin conforming layer and an outer layer, the outer layer is adjacent the skin conforming layer, wherein the skin conforming layer is in contact with the skin and comprises a hydrophilic hydrogel, and the outer layer includes a hydrophobic polymer.

18. The bionic dressing of claim 17, wherein the bionic dressing is formed by the following process:
mixing the oil phase and the aqueous phase to form a mixture comprising the oil phase and the aqueous phase,
applying the mixture to a surface to form an uncured bionic dressing, and
irradiating the uncured bionic dressing with light to form the bionic dressing.

19. A method of forming a bionic dressing, wherein the method comprises:
applying a mixture comprising the aqueous phase and the oil phase of the bionic dressing-forming composition of claim 1; and
curing the mixture via photopolymerization initiated by light, thereby obtaining the bionic dressing.
